(19) United States
(12) Patent Application Publication
(10) Pub. No.: US 2023/0404870 A1
(43) Pub. Date: Dec. 21, 2023

(54) A FLUORIDE DENTIFRICE CONTAINING AN IODINE COMPONENT

(71) Applicants: ADVANTAGE SILVER DENTAL ARREST, LLC, Salem, OR (US); ELEVATE ORAL CARE, LLC, West Palm Beach, FL (US)

(72) Inventor: Peter M. Milgrom, Seattle, WA (US)

(73) Assignees: ADVANTAGE SILVER DENTAL ARREST, LLC, Salem, OR (US); ELEVATE ORAL CARE, LLC, West Palm Beach, FL (US)

(21) Appl. No.: 18/251,964

(22) PCT Filed: Nov. 11, 2021
(86) PCT No.: PCT/US21/58962

§ 371 Date: May 5, 2023

Related U.S. Application Data
(60) Provisional application No. 63/114,474, filed on Nov. 16, 2020.

(51) Int. Cl. A61K 8/20; A61K 8/81; A61K 8/21; A61Q 11/00

(52) U.S. Cl. CPC A61K 8/20; A61K 8/8176; A61K 8/21; A61Q 11/00; A61K 2800/28; A61K 2800/54; A61K 2800/412

(57) ABSTRACT

An oral dentifrice composition containing polyvinyl pyrrolidone-iodine (PVP-I) and a fluoride component at a concentration to provide at least 4500 ppm fluoride to the oral dentifrice composition. The composition provides for increased uptake of fluoride in enamel as well as having improved antibacterial and antiseptic properties over other dentifrices that 5 do not contain PVP-I. Also, disclosed is a method for the treatment or prophylaxis of caries comprising applying the oral dentifrice to the teeth.

### TECHNICAL FIELD

The present invention is drawn to a prescription strength fluoride dentifrice (paste, gel, cream or powder) composition comprising polyvinyl pyrrolidone-iodine (PVP-I; povidone iodine), and optionally an abrasive, that provides for increased uptake of fluoride in enamel as well as having improved antibacterial and antiseptic properties over other dentifrices that do not contain PVP-I. The present invention is also drawn to a method of the treatment or prophylaxis of caries comprising applying the prescription strength fluoride dentifrice to the teeth.

### BACKGROUND OF INVENTION

Products of metabolism by bacteria populating the tooth surface induce development and progression of caries lesion (cavities). Dental caries is one of the most common diseases in the world. It is also a preventable disease if proper care is taken. Individuals with higher rates of tooth decay have higher concentrations of pathogenic bacteria colonizing the teeth than patients with low tooth decay rates. Streptococcus mutans is the primary pathogen associated with tooth decay; it and other organisms produce acids that demineralize the teeth resulting in cavities.

Fluoride dentifrices have traditionally been available for patients for home use. Dentifrices containing 850 to 1100 ppm fluoride products in a variety of fluoride forms are available over the counter and are effective at preventing decay. U.S. Pat. No. 5,156,835 (Colgate Palmolive Co.) teaches dentifrices comprising about 1000-1500 ppm of fluoride from either sodium fluoride or sodium monofluorophosphate. These dentifrices include an antibacterial antiplaque agent of 2,4,4′-trichloro-21-hydroxydiphenyl ether (triclosan) or hexyl resorcinol and a synthetic anionic linear polymeric polycarboxylate having a molecular weight of about 1,000 to about 1,000,000 to inhibit plaque formation. However, the U.S. Food and Drug Administration has taken the position that certain products can no longer be marketed containing triclosan.

U.S. Pat. No. 5,843,408 discloses a povidone-iodine semi-paste oral preparation comprising at least from about 0.1 to about 20% by weight of povidone-iodine, about 0 to about 50 parts by weight of potassium iodide and from about 1 to about 300 parts by weight of a pharmaceutically stable base comprising a substantially fully reduced oligosaccharide. The povidone-iodine is added for its microbicidal or antiseptic properties. Among a long list of possible additives, this patent teaches that an effective amount of a pharmaceutically acceptable fluoride ion can be added. There is no teaching of how much fluoride should be used.

Thus, there are problems with prior art fluoride compositions containing antimicrobial components, and there is a need for improved fluoride compositions in the treatment and prophylaxis of dental caries.

### SUMMARY OF THE INVENTION

The present invention is drawn to a dentifrice (paste, gel, cream or powder) composition comprising at least 4500 ppm fluoride and an antimicrobial amount of polyvinyl pyrrolidone-iodine (PVP-I; povidone iodine), and optionally an abrasive, that provides for increased uptake of fluoride in enamel as well as having improved antibacterial and antiseptic properties over other dentifrices that do not contain PVP-I. The inventive dentifrice can remove plaque, while preventing caries by synergistically remineralizing the tooth structure and reducing the pathogenic bacterial load in the oral environment.

The higher concentration of fluoride has more efficacy when compared to compositions having a low concentration of fluoride, as fluoride efficacy has a concentration-response relationship. The fluoride causes the formation of calcium fluoride which serves as a fluoride reservoir to promote remineralization of lesions and prevent them forming in other teeth in the mouth. Fluoride acts to remineralize demineralized or softened areas of tooth structure. In addition, the optional abrasives in the dentifrice and the brushing action by the user can act to physically disrupt microbial plaque. The fluoride itself is not expected to have practical biological effectiveness against the pathogen organisms because of the short exposure with the dentifrice. As such, the inventive dentifrice includes the PVP-I in an effective concentration to have the antimicrobial effects.

In a first aspect, the disclosure relates to an oral dentifrice composition comprising polyvinyl pyrrolidone-iodine (PVP-I) and a fluoride component at a concentration to provide at least 4500 ppm fluoride to the oral dentifrice composition.

In the foregoing embodiment, the PVP-I may be present in an amount of about 0.5 wt. % to about 15 wt. %, or about 2 wt. % to about 15 wt. %, or about 7 wt. % to about 12 wt. % based on a total amount of the oral dentifrice composition.

In each of the foregoing embodiments, the PVP-I may provide iodine in an amount of about 0.05 wt. % to about 1.5 wt. %, or about 0.2 wt. % to about 1.5 wt. %, or about 0.7 wt. % to about 1.2 wt. % based on a total amount of the oral dentifrice composition.

In each of the foregoing embodiments, the fluoride component may be a fluoride salt.

In each of the foregoing embodiments, the fluoride component may be sodium fluoride (NaF), BiF₃, SnF₂, ZnF₂, KF, CaF₂, ZrF₄, sodium mono-fluorophosphate (Na₂FPO₃), hexafluorosilicic acid (H₂SiF₆), sodium hexafluorosilicate (Na₂SiF₆), or combinations thereof.

In each of the foregoing embodiments, the fluoride component may be included in the composition in an amount from about 0.05 wt. % to about 10 wt. %, or about 0.1 wt. % to about 10 wt. % or about 0.9 wt. % to about 3 wt. %, or about 1.1 wt. % based on a total amount of the oral dentifrice composition

In each of the foregoing embodiments, the fluoride component may be included in the composition in an amount sufficient to provide at least 4600 ppm fluoride, 4500 ppm to 7000 ppm fluoride, 4600 ppm to 6000 ppm fluoride, 4700 ppm to 5500 ppm fluoride, or 4800 ppm to 5250 ppm fluoride to the oral dentifrice composition, based on a total amount of the oral dentifrice composition.

In each of the foregoing embodiments, the fluoride component may be sodium fluoride.

In each of the foregoing embodiments, the fluoride component may be sodium fluoride and may have an average particle size (diameter) of less than 50 micrometers (microns), or at least 80% of all the particles have a diameter of less than 16 microns, as measured by sieve analysis.

In each of the foregoing embodiments, the fluoride component may be sodium fluoride where all or nearly all (such as greater than 90%, 95%, or 99%) of the fluoride particles are less than 20±2 microns in diameter.

In each of the foregoing embodiments, the oral dentifrice may be a paste, gel, cream, or powder, preferably a gel or paste.

In each of the foregoing embodiments, the oral dentifrice may further comprise an abrasive agent.

In each of the foregoing embodiments, the oral dentifrice may further comprise an abrasive agent selected from hydrated silica; alumina (including calcined aluminum oxide); diatomaceous earth; pumice; calcium carbonate; cuttlebone; insoluble phosphates, including orthophosphates, polymetaphosphates and pyrophosphates. Illustrative examples are dicalcium orthophosphate dihydrate, dicalcium phosphate dihydrate, calcium hydrogen phosphate, calcium pyrophosphate, β-calcium pyrophosphate, tricalcium phosphate, calcium metaphosphate, potassium metaphosphate, and sodium metaphosphate; composite resins, such as melamine resin, phenolic resin, and urea-formaldehyde resin and polycarbonate; boron carbide; microcrystalline wax; and microcrystalline cellulose including combinations of colloidal microcrystalline cellulose and carboxymethylcellulose, and combinations and derivatives thereof, preferably, the abrasive agent is in an amount of about 0.5 to about 50 wt. %, or 2 to 25 wt. % or 5 to 20 wt. % based on the total weight of the oral dentifrice composition.

In each of the foregoing embodiments, the oral dentifrice may further comprise a tooth whitening or tooth bleaching component selected from peroxides including hydroperoxides, hydrogen peroxide, peroxides of alkali and alkaline earth metals, organic peroxy compounds, peroxy acids, perborate, and urea peroxide; metal chlorites including calcium chlorite, barium chlorite, magnesium chlorite, lithium chlorite, sodium chlorite, and potassium chlorite; and persulfates.

In each of the foregoing embodiments, the tooth whitening or tooth bleaching component may be included in an effective amount, or from 1% to 20% by weight based on the total weight of the oral dentifrice composition.

In each of the foregoing embodiments, the oral dentifrice may further comprise one or more of potassium phosphate, a pH adjusting agent, surfactant, flavoring agent or sweetener, additional active ingredients, antibacterial agents, enzymes, an orally acceptable carrier, colorant, anticalculus agent, foaming agent, solubilizing agent, cleansing agent, humectant, antisensitivity agents, abrasive agent, tooth whitening or tooth bleaching component, and a hydrating agent.

In each of the foregoing embodiments, the oral dentifrice may include one or more of the following:
a) A solvent, such as water to dissolve ingredients;
b) A hydrating agent, such as sorbitol or glycerin;
c) An abrasive agent, such as hydrated silica;
d) A humectant, such as PEG-12 or PEG-400;
e) A pH adjusting agent, such as one or more of tetrasodium pyrophosphate, sodium citrate and citric acid;
f) A solubilizing or cleansing agent, such as sodium lauryl sulfate;
g) A flavoring agent, such as one or more of sodium saccharin and sucralose;
h) A thickener or stabilizer, such as one or more of microcrystalline cellulose, cellulose gum, xanthan gum and zinc phosphate;
i) A phosphate, such as potassium phosphate monobasic;
j) A foaming agent or thickener such as cocamidopropyl betaine; and
k) A colorant such as titanium dioxide.

In each of the foregoing embodiments, the oral dentifrice may not contain saccharides.

In a second aspect, the disclosure relates to a method for the treatment or prophylaxis of dental caries comprising applying the oral dentifrice composition of each of the foregoing embodiments to teeth in a mammal, or a human.

Additional features of the present disclosure will become apparent to those skilled in the art upon consideration of the following detailed description of the presently perceived best mode of carrying out the disclosure.

### BRIEF DESCRIPTION OF THE DRAWINGS

FIG. 1 shows a plot of the synergistic effect of the proportion of PVP-I and NaF.

### DETAILED DESCRIPTION OF THE INVENTION

### Definitions

The following definitions of terms are provided in order to clarify the meanings of certain terms as used herein.

The term “dental caries”, commonly referred to as tooth decay, is a disease in which damage to the tooth structure occurs. The damaged tooth structure is called a cavity or dental caries lesion that is caused by acid release from the bacteria colonizing the tooth surface. The tooth includes, in part, the enamel, the dentin, cementum, and the pulp. The enamel comprises the outer surface of the crown of the tooth, and the dentin is the layer just below the enamel. The cementum covers the root surface. The pulp is the central part of the tooth, which includes soft connective tissue, blood vessels and nerves. Dental caries, as used herein, refers to destruction or decay of the enamel, dentin, cementum and/or pulp or any combination thereof. Carious lesions refer to injury to the tooth structure that is caused by dental caries.

The presence, absence, or state of caries lesions can be determined by a health professional or lay person using methods that are known in the art. For example, early dental caries is determined by a visual identification of “white spot” lesions. Caries lesions are also determined by visual and tactile exam identifying discolored or decalcified pits and fissures. Frank cavitation is identified as a clear break in the enamel. The presence of white spots discolored or decalcified pits and fissures, or frank cavitation indicates the presence of dental caries. Inspection of visible tooth areas can be performed with a dental mirror and explorer. Caries can be identified by its texture and architecture. Healthy enamel and dentin are denser to probing with a dental instrument, i.e., dental explorer, as compared to enamel and dentin that are infected with dental caries. Additionally, caries lesions can be diagnosed with use of X-rays, especially in areas that are not easily visible. Other technologies such as fiber optic illumination, lasers, and dyes can also be used to identify the presence or absence of dental caries lesions.

The present invention further includes treating dental caries. “Treating” dental caries refers to the prevention or cessation or reduction of progression of caries lesions. Treating dental caries includes preventing the carious lesion from beginning or getting worse. For example, the carious lesion is treated when the lesion does not get larger in size and/or does not further affect additional tooth structure (e.g., penetrate from the enamel to the dentin).

As used herein, the term “effective amount” means an amount of a composition comprising the antimicrobial material PVP-I, that is effective to prevent or arrest formation of dental caries. Such a composition may also include one or more additional active ingredients, including without limitation one or more inactive ingredients, as discussed below.

An “orally acceptable carrier” as used herein means a material or materials which are used to apply the compositions of the present invention to the oral cavity in a safe and effective manner.

As used herein, “cleaning” generally refers to the removal of contaminants, dirt, impurities, and/or extraneous matter on a target surface. For example, in the context of oral surfaces, where the surface is tooth enamel, the cleaning may remove at least some of a film or stain, such as plaque biofilm, pellicle or tartar.

### Dentifrice

### Oral Dentifrice Composition

The oral dentifrice composition can be in the form of a paste, gel, cream or powder.

Preferably, the oral dentifrice composition is in a paste or gel form. Rigidity and viscosity are two separate rheological parameters used to characterize the mechanical properties of the present invention. Preferably, the paste or gel compositions of the present invention possess the following properties:
1. Uniformity: the composition should be formulated so that it is and can remain uniform without separation or precipitation over time.
2. Flowability: The composition, when placed in a tube or container and expelled under shear force should be flowable.
3. Stability/Breakability: The fine balance between stability and breakability of the paste or gel coming out of the tube or container is very delicate: on the one hand, the gel should preferably not be very runny upon release from the tube or container and not lose its thixotropy property as a result of exposure to a tooth; and on the other hand, it should be “breakable”, i.e., it should spread easily, break down and absorb onto the surface upon application of mild shear force.

The present invention has been made in view of the above circumstances, and it is an object of the present invention to provide an oral composition having suitable viscosity and thixotropic property for use on an interdental brush.

The inventive composition for oral application may have a viscosity of from 0.02 to 100 Pa·s, as measured by a Brookfield Viscometer at 30° C., at 20 rpm. One skilled in the art would know to use an appropriate spindle, RV2, RV3, RV4, or RV6, depending on the viscosity of the emulsion being tested.

In each of the foregoing embodiments, a thickener or stabilizer may be used. Suitable examples of the thickener or stabilizer may be selected from cellulose derivative (“cellulose gum”) such as carboxymethyl cellulose (CMC), methyl cellulose, hydroxyethyl cellulose, hydroxypropyl methyl cellulose, microcrystalline cellulose, and mixtures thereof; polyvinyl pyrrolidone; xanthan; an admixture of glycerin and polyacrylate; carrageenans such as iota-carrageenan, kappa-carrageenan, kappa2-carrageenan, lambda-carrageenan, and mixtures thereof; guar gum; gum karaya; gum arabic; gum tragacanth; and mixtures thereof, hydrated silica and colloidal silica may be used as thickeners, or silica thickeners. Preferably, the thickener or stabilizer is a carboxymethyl cellulose. The thickener or stabilizer may be present in an effective amount, e.g., 0.1 to 15 wt. %, or 0.25 to 10 wt. %, or 0.5 to 5 wt. %, based on the total weight of the oral dentifrice composition.

In each of the foregoing embodiments, the thickener or stabilizer may act as a thixotropic additive. Thixotropic additives are capable of integrating a compound that is capable of polymerizing or that has already partly or fully, polymerized, and has at least two groups available for hydrogen bonding, into a gel-like three-dimensional network.

Thixotropic additive will impact a composition to have time-dependent shear thinning properties. Therefore, the oral composition may be thick or viscous under static conditions, however the composition may flow (become thinner, less viscous) over time when shaken, agitated, shear-stressed, or otherwise stressed. This property enables application of a thixotropic mixture as a semi-solid state to a body surface, which subsequently becomes substantially liquid and therefore more spreadable when applied to a surface, for example, on a tooth.

More specifically, when the composition of the present invention is sheared by squeezing the product bottle, the weak chemical bonds are broken, and a lyophobic solution is formed that can be applied to the decayed tooth. Once applied to the tooth, the particles collide, flocculation occurs, and the gel is reformed such that the dental provider can control the application.

In each of the foregoing embodiments, the composition of the present invention may be thixotropic, and thus capable of undergoing an isothermal gel-sol-gel transformation. In one or more embodiments, when poured, it displays flow, but over time it reverts to being more viscous or gel like. In one or more embodiments, when shear force is applied it displays flow, but overtime reverts to being more viscous or gel like. In one or more embodiments, a solid gel becomes flowable and later with time become solid or semi-solid. In one or more embodiments a semi-solid gel becomes flowable and later with time become solid or semi solid. In one or more embodiments, a liquid gel is flowable and later with time becomes solid or semi solid. In each of the foregoing embodiments, the oral dentifrice composition may have a viscosity of greater than 75 Pa·s, or greater than 100 Pa·s, or greater than 125 Pa·s at a shear rate of 0.1/sec and less than 15 Pa·s, or less than 10 Pa·s, or less than 8 Pa·s at a shear rate of 100/sec.

In each of the foregoing embodiments, thickener or stabilizers, which may also act as thixotropic additives may be selected from carboxymethyl cellulose, carrageenan, and silica. Thickener or stabilizers, such as xantham gum typically do not impart thixotropy properties when added to compositions. When a thixotropic additive is included in the compositions of the present invention, the composition may have a thixotropy index of from 0.3 to 0.65, wherein the thixotropy index (“TI”) is calculated by formula (I):

$$TI = log 10 ( \frac{M 5}{M 50} ) = 0.3 to 0.65$$

wherein M5 represents an apparent viscosity of the oral composition as measured by a Brookfield viscometer at 5 rpm and 30° C., and M50 represents an apparent viscosity of the oral composition as measured by a Brookfield viscometer at 50 rpm and 30° C.

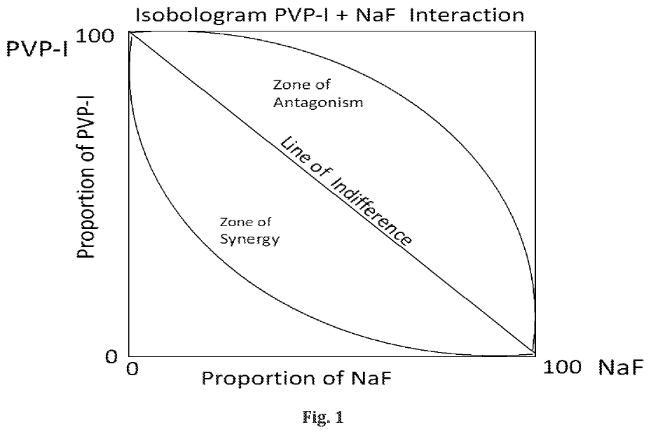

The inventive composition is a dentifrice, not a varnish. A varnish is a liquid preparation that when applied to the surface of the tooth dries to form a transparent coating. Prior to application of the varnish, the surface of the teeth are dried. The varnish has a low viscosity, typically between 30-60 centipoise, and is applied with a varnish applicator (see FIG. 1 of U.S. Pat. No. 9,107,838 B2 for an example of an applicator). The varnish is applied by a dentist. On the other hand, the dentifrice has a suitable viscosity and thixotropic property for use on an interdental brush. The dentifrice is used in the same fashion as toothpaste. The user applies the dentifrice to the top of the bristles of the interdental brush and then while holding the handle of the interdental brush, cleans the areas of the mouth. There is no need for drying the teeth prior to use of the dentifrice. This cleaning with the dentifrice can be done by either the dentist or the user.

### Additional Additives

In each of the forgoing embodiments, the oral dentifrice composition may include one or more of potassium phosphate, a pH adjusting agent, surfactant, flavoring agent or sweetener, additional active ingredients, antibacterial agents, enzymes, an orally acceptable carrier, colorant, anticalculus agent, foaming agent, solubilizing agent, cleansing agent, humectant, antisensitivity agents, abrasive agent, tooth whitening or tooth bleaching component, a hydrating agent or the like.

In each of the foregoing embodiments, the oral dentifrice composition may comprise potassium phosphate (like potassium phosphate monobasic). This additive can be included to boost the fluoride uptake in the tooth. Calcium additives have been used commercially to improve remineralization of teeth; however, calcium can bind up fluoride if not properly used. In a preferred embodiment of the present invention, there is no source of calcium in the composition. In the place of the source of calcium, the phosphate is used to provide yet another building block of tooth enamel and dentin. The oral dentifrice composition may comprise potassium phosphate in effective amounts, e.g., from an amount of 0.1 wt. % to 2 wt. %, preferably about 0.5 wt. %, based on the total weight of the oral dentifrice composition.

In each of the foregoing embodiments, the oral dentifrice composition may comprise a pH adjusting agent. Examples of the pH adjusting agent include, for example, citric acid, phosphoric acid, malic acid, pyrophosphoric acid, lactic acid, tartaric acid, glyceric phosphoric acid, acetic acid, and nitric acid, or a chemically available salt thereof. The pH can be controlled with acid (e.g. citric acid or benzoic acid) or base (e.g. sodium hydroxide) or buffered (as with sodium citrate, benzoate, carbonate, or bicarbonate, disodium hydrogen phosphate, sodium dihydrogen phosphate, etc.). Sodium hydroxide or the like can be blended alone or in combination of two or more so that the pH of the liquid oral composition of the present invention is in the preferred range. In each of the foregoing embodiments, the pH of the oral dentifrice composition can be in the range of pH in the range from 1.5 to 7.5, preferably from 4.5 to 6.5, or about 6. The pH adjusting agent can be used in an amount of 0.001 to 10.0% by weight based on the total weight of the oral dentifrice composition.

In each of the foregoing embodiments, the oral dentifrice composition may comprise a surfactant. Surfactants include nonionic surfactants such as propylene dalicol fatty acid ester, glycerin fatty acid ester, polyglycerin fatty acid ester, sorbitan fatty acid ester, polyoxyethylene sorbitan fatty acid ester, and polyoxyethylene sorbite fatty acid ester, polyoxyethylene hydrogenated castor oil, poly (ethylene glycol) fatty acid ester, polyoxyethylene alkyl ether, polyoxyethylene polyoxypropylene glycol, etc., and betaine type, imidazoline type as amphoteric surfactants, amide-type surfactants as cationic surfactants, lecithin derivatives, alkyl phosphates, polyoxetylene alkyl ether phosphates as sodium anionic surfactants. N-acyl examples include amino acid salts, persyl taurine salts, alkyl ether carboxylates, sulfonates, alkyl sulfates, and polyoxyethylene alkyl ether sulfates. These may be used singly or in combination of two or more. The surfactant may be added in effective amounts, e.g., from 0.01 to 10% by weight based on the total weight of the oral dentifrice composition. When using less than 0.01 wt. % and more than 10 wt. % of surfactant there can be a negative effect on the mouth feel, fluoride uptake or release, foaming action, and plaque removal.

In each of the foregoing embodiments, the oral dentifrice composition may comprise a flavoring agent. Suitable flavoring agents may be selected from, but are not limited to, essential oils, as well as various flavoring aldehydes, esters, alcohols, and similar materials. Examples of essential oils include oils of spearmint, peppermint, wintergreen, sassafras, clove, sage, eucalyptus, marjoram, cinnamon, lemon, lime grapefruit, and orange. The flavoring agents include menthol, carvone, anethole, eugenol, methyl salicylate, limonene, ocimene, n-decyl alcohol, citronellol, α-terpineol, methyl acetate, citronellyl acetate, methyl eugenol, rosemary oil, pimento oil, and perilla oil. The flavoring agent may be added in effective amounts, e.g., from approximately 0.1 to about 5.0 wt. % by weight, or from about 0.5 to about 1.5 wt. %, based on the total weight of the oral dentifrice composition.

Further, as sweeteners, saccharin sodium, stevioside, neohesperidyl dihydrochalcone, glycyrrhizin, thaumatin, aspartyl phenylalanine methyl ester, ρ-methoxycinnamic aldehyde, xylitol, and palatinose may be used. Examples thereof include palatinit, erythritol, maltitol, and the like. These can be used alone or in combination of two or more. The sweeteners may be added in effective amounts, e.g., is preferably from about 0.01 to about 5 wt. % based on the total weight of the oral dentifrice composition.

In each of the foregoing embodiments, the oral dentifrice composition may comprise an active ingredient in addition to the fluoride. The active ingredients include for example, anti-tartar agents, anti-caries agents, anti-inflammatory agents, anti-sensitivity agents, nutrients, and the like. Actives useful herein are optionally present in the compositions of the present invention in safe and effective amounts that are sufficient to have the desired therapeutic or prophylactic effect in the human or lower animal subject to whom the active is administered, without undue adverse side effects (such as toxicity, irritation, or allergic response), commensurate with a reasonable risk/benefit ratio when used in the manner of this invention. The specific safe and effective amount of the active will vary with such factors as the particular condition being treated, the physical condition of the subject, the nature of concurrent therapy (if any), the specific active used, the specific dosage form, the carrier employed, and the desired dosage regimen. The active ingredients include for example vitamins such as d1-α-tocopherol acetate, tocopherol succinate, or tocopherol nicotinate; amphoteric fungicides such as diaminoethyl glycine; nonionics such as isopropylmethylphenol; enzymes; alkali metal monofluorophosphates such as sodium monofluorophosphate, and potassium monofluorophosphate; tranexamic acid; epsilon aminocaproic acid; allantoin; dihydrocholesterol, glycyrrhizinates, glycyrrhetinic acid, glycerophosphate, sodium chloride, water-soluble inorganic phosphate compounds, etc. Such active ingredients may be added in effective amounts, e.g., from 0.5 to 5 wt. % based on the total weight of the oral dentifrice composition.

In each of the foregoing embodiments, the oral dentifrice composition may also include one or more antibacterial agents in addition to the PVP-I. Antibacterial agents are known in the art, and include benzoic acid, sodium benzoate, potassium benzoate, boric acid, and phenolic compounds such as betanaphthol, chlorothymol, thymol, anethole, eucalyptol, carvacrol, menthol, phenol, amylphenol, hexylphenol, heptylphenol, octylphenol, hexylresorcinol, laurylpyridinium chloride, myristylpyridinium chloride, cetylpyridinium fluoride, cetylpyridinium chloride, and cetylpyridinium bromide. Compositions of the present invention may also include one or more basic amino acids, e.g., arginine, in free base or salt form. Such agents may be added individually or in combination in effective amounts, e.g., from 1% to 20% by weight based on the total weight of the oral dentifrice composition, depending on the agent chosen.

In each of the foregoing embodiments, the oral dentifrice composition may also include one or more enzymes. Useful enzymes include any of the available proteases, glucanohydrolases, endoglycosidases, amylases, mutanases, lipases and mucinases or compatible mixtures thereof. In certain embodiments, the enzyme is a protease, dextranase, endoglycosidase and mutanase. In another embodiment, the enzyme is papain, endoglycosidase or a mixture of dextranase and mutanase. An enzyme or a mixture of several compatible enzymes may be added in effective amounts, e.g., from 0.002 wt. % to 2.0 wt. %, or 0.05 wt. % to 1.5 wt. %, or 0.1 wt. % to 0.5 wt. % based on the total weight of the oral dentifrice composition.

An orally acceptable carrier may also be present in the oral dentifrice compositions of each of the foregoing embodiments of the invention. An orally acceptable carrier is preferably water. The water can be deionized and free of organic impurities. Water commonly makes up the balance of the oral dentifrice and includes 10% to 90%, 20% to 60% or 10% to 30% by weight of the oral dentifrice compositions. This amount of water includes the free water which is added plus that amount which is introduced with other materials such as with sorbitol or any components of the invention.

In addition, colorants can be added to improve the appearance of each of the foregoing embodiments of the oral dentifrice composition. Colorants herein include pigments, dyes, lakes and agents imparting a particular luster or reflectivity such as pearling agents. Any orally acceptable colorant can be used, including without limitation talc, mica, magnesium carbonate, magnesium silicate, magnesium aluminum silicate, titanium dioxide, red, yellow, brown and black iron oxides, ferric ammonium ferrocyanide, manganese violet, ultramarine, titaniated mica, bismuth oxychloride, and the like. One or more colorants may be added in effective amounts, e.g., from 0.001 wt. % to 20 wt. %, for example, from 0.01 wt. % to 10 wt. %, or from 0.1 wt. % to 5 wt. %, based on the total weight of the oral dentifrice composition.

An anticalculus agent may also be provided to each of the foregoing embodiments of the oral dentifrice composition by the inclusion of a molecularly dehydrated polyphosphate salt. The linear molecularly dehydrated polyphospate salts operative herein as anticalculus agents are generally employed in the form of their wholly or partially neutralized water soluble alkali metal (e.g. potassium and preferable sodium) or ammonium salts, and any mixtures thereof. Representative examples include sodium hexametaphosphate, sodium tripolyphosphate, disodium diacid, trisodium monoacid and tetrasodium pyrophosphates and the like. Linear polyphosphates correspond to (NaPO₃))n where n is about 2 to about 125 The anticalculus agent may be added in effective amounts, e.g., from of 0.1 to 7 wt. % preferably 1 to 7 wt. %, more preferably 2 to 7 wt. % based on the total weight of the oral dentifrice composition. When n is at least 3 in (NaPO₃)n, the polyphosphates are glassy in character.

Particularly desirable anticalculus agents are tetraalkali metal pyrophosphates, including mixtures thereof, such as tetrasodium pyrophosphate tetrapotassium, pyrophosphate and mixtures thereof. An anticalculus agent comprising about 4.3% to about 7% by weight based on the total weight of the oral dentifrice composition wherein the weight ratio of tetrapotassium pyrophosphate to tetrasodium pyrophosphate is from about 4.3:2.7 to about 6:1 is especially preferred.

In each of the foregoing embodiments, the oral dentifrice composition may also include one or more foaming agents that optionally also act as solubilizing or cleansing agents may include sodium laurylsulfate, sodium α-olefinsulfates, N-acylglutamates, N-acyltaurates, sucrose fatty acid esters, polyoxyethylene hydrogenated castor oil, cocamidopropyl betaine and polyglycerin fatty acid esters and the like. One or more foaming agents may be added in effective amounts, e.g., from 0 wt. % to 5 wt. %, for example, from 0.1 wt. % to 4 wt. %, or from 0.5 wt. % to 1.5 wt. %, based on the total weight of the oral dentifrice composition.

In each of the foregoing embodiments, compositions of the present invention may also comprise a humectant, e.g., to prevent the composition from hardening upon exposure to air. Certain humectants can also impart desirable sweetness or flavor to dentifrice compositions. The humectant, on a pure humectant basis, generally includes 5 wt. % to 70 wt. % in one embodiment or 30 wt. % to 65 wt % in another embodiment based on the total weight of the oral dentifrice composition. Suitable humectants include edible polyhydric alcohols such as glycerine, sorbitol, xylitol, propylene glycol, PEG-12 or PEG-400 as well as other polyols and mixtures of these humectants. Mixtures of glycerine and sorbitol may be used in certain embodiments as the humectant component of oral dentifrice compositions herein.

In each of the foregoing embodiments, the oral dentifrice composition may also include one or more antisensitivity agents, e.g., potassium salts such as potassium nitrate, potassium bicarbonate, potassium chloride, potassium citrate, and potassium oxalate; capsaicin; eugenol; strontium salts; zinc salts; chloride salts and combinations thereof. Such agents may be added in effective amounts, e.g., from 1 wt % to 20 wt % by weight based on the total weight of the oral dentifrice composition, depending on the agent chosen. The compositions of the present invention may also be used to treat hypersensitivity by blocking dentin tubules when applied to a tooth.

In each of the foregoing embodiments, the oral dentifrice composition may also include one or more abrasive agents selected from hydrated silica; alumina (including calcined aluminum oxide); diatomaceous earth; pumice; calcium carbonate; cuttlebone; insoluble phosphates, including orthophosphates, polymetaphosphates and pyrophosphates. Illustrative examples are dicalcium orthophosphate dihydrate, dicalcium phosphate dihydrate, calcium hydrogen phosphate, calcium pyrophosphate, β-calcium pyrophosphate, tricalcium phosphate, calcium metaphosphate, potassium metaphosphate, and sodium metaphosphate; composite resins, such as melamine resin, phenolic resin, and urea-formaldehyde resin and polycarbonate; boron carbide; microcrystalline wax; and microcrystalline cellulose including combinations of colloidal microcrystalline cellulose and carboxymethylcellulose, and combinations and derivatives thereof. The abrasive agent may be added in effective amounts, e.g., from 0.5 to 50 wt. %, or 2 to 25 wt. % or 5 to 20 wt. % based on the total weight of the oral dentifrice composition.

In each of the foregoing embodiments, the oral dentifrice composition may not contain plastic microbeads. These microbeads are bad for the environment.

In each of the foregoing embodiments, the oral dentifrice composition may also include one or more tooth whitening or tooth bleaching components selected from peroxides including hydroperoxides, hydrogen peroxide, peroxides of alkali and alkaline earth metals, organic peroxy compounds, peroxy acids, perborate, and urea peroxide; metal chlorites including calcium chlorite, barium chlorite, magnesium chlorite, lithium chlorite, sodium chlorite, and potassium chlorite, and persulfates. The tooth whitening or tooth bleaching component can be included in an effective amount, e.g., from 1% to 20% by weight based on the total weight of the oral dentifrice composition

The container for filling the oral dentifrice composition of the present invention may be any container that can be easily extruded and can discharge an appropriate amount to the interdental brush. For example, tubes such as aluminum tubes and laminate tubes, or materials such as polyethylene terephthalate, polyethylene, polypropylene, polyfluorocarbon, polychlorinated vinyl, polyatarylate, ethylene-vinyl alcohol polymer, and ataryl nitrile copolymer Bottle containers can be used.

In another aspect, the present invention relates to a method of treatment or prophylaxis of dental caries comprising applying the oral dentifrice composition of any of the foregoing embodiments to teeth in a mammal, or a human.

### Example 1

The following examples are illustrative, but not limiting, of the methods and compositions of the present disclosure. Other suitable modifications and adaptations of the variety of conditions and parameters normally encountered in the field, and which are obvious to those skilled in the art, are within the spirit and scope of the disclosure. All patents and publications cited herein are fully incorporated by reference herein in their entirety.

A toothpaste composition capable of being squeezed from a tube and able to stably rest on the bristles of a toothbrush is formed by combining sodium fluoride in an amount sufficient to make up 1.1% wt. % of the composition, 10% wt. % PVP-I, sorbitol, deionized water, hydrated silica, xylitol, glycerin, PEG 600, sodium lauryl sulfate, potassium phosphate monobasic, titanium dioxide (color-masking agent), flavor, and sodium saccharin.

### Stability:

The toothpaste packaged in sterile containers is subjected to a 1-mo accelerated test at 400° C./75% relative humidity. The toothpaste is assessed for pre/post weight loss) and evaluated qualitatively for stability of viscosity, color, odor and taste.

The formulations containing (1-10% PVP-I with 5% NaF) are thoroughly mixed and then standardized aliquots taken and tested to verify the availability of both iodine and fluoride. Release of iodine's species is determined by inductively coupled plasma-mass spectrometry. The details of sample preparation, columns, and detection of species of iodine can be found in Lin et al. 2018. For fluoride, the ion-specific electrode method for quantification is used. Electrodes are calibrated to assure standardization and linear response within concentration range of samples. Results are plotted as a function of time—f(time)—to characterize the release of F− from the test products, and t-tests assess any difference in mean F− released at each time point. AUC is calculated and the total F− recovered is compared amongst the test products: a) the inventive composition comprising 5 or 10% PVP-I including 5% NaF; b) a comparative sample containing the standard 5% NaF toothpaste; and c) a control toothpaste to which no NaF or PVP-I is added.

### Synergism:

There are several methods to determine whether 2 antibacterial agents work with synergy. It is expected that the inventive composition has a synergistic effect, and thus can be used to effect at lower doses and consequently, lower toxicities.

To examine for the synergistic effect between the 2 anti-septic agents of PVP-I and fluoride delivered by a novel toothpaste topically, the method involves the initial identification of the potency of the individual agents which, for the sake of simplicity, we will refer to as A and B—to identify their maximal effect singly, per se the killing of the target microorganism or the inhibition of its growth. The agents of interest, neither of which are prominently toxic, inhibit the growth of the bacteria that cause caries. One of them, fluoride, also has effects on the de-mineralization/re-mineralization of enamel; as will be addressed in the method below.

To investigate whether those agents can plausibly be used at lower concentrations with near-maximal or even superior (synergistic) effectiveness and, consequently, with possibly substantial reduction of doses, a series of concentrations are studied of each agent individually released from toothpaste with the goal of experimentally determining each agent's maximal effect alone on the inhibition of the target microorganism's (S. mutans) growth. Concurrently, a series is prepared of proportional combinations of these agents (thus, 100 parts A/0 parts B; 90 parts A/10 parts B; 80 parts A/20 parts B; 70 parts A/parts 30 B; 60 parts A/40 parts B; 50 parts A/50 parts B; 40 parts A/parts 60 B; 30 parts A/70 parts B; 20 parts A/80 parts B; 10 parts A/parts 90 B; and 0 parts A/100 parts B). In this way, it can be discriminated between whether the effects of the two agents against S. mutans would not just be additive in growth inhibiting effect but decidedly synergistic in growth inhibition. If that proves to be the case, those lower concentrations could be used to optimize the effect.

The plausibility of synergy between PVP-I and NaF is based on the fact that both agents have antibacterial actions that differ. In the case of S. mutans, NaF is bacteriostatic, it doesn't kill these cells; but it inhibits their growth. PVP-I, however, kills many bacteria, fungi, and even some viruses, by interfering with their lipid structures and metabolism. Thus, while their mechanisms of actions are decidedly different, their concurrent action could well not only be additive but also synergistic. Both of these agents are remarkably well-tolerated in the mouth.

### Procedures:

A modification of the classical Kirby-Bauer disc diffusion technique is used to study inhibition of S. mutans growth. The method involves the seeding of “lawns” of freshly grown S. mutans onto nutrient agar plates (either trypticase-soy or brain-heart infusion agars supplemented with glucose serve well) to support luxuriant growth of these bacteria upon incubation at 37° C. in an atmosphere of air supplemented with CO₂, much as exists on the surface of teeth. The seeding of even lawns is accomplished by pipetting 0.5 mL of log phase broth inoculum culture onto agar plates, placing them onto a rotating laboratory turntable, and spreading the inocula with a sterile bent glass rod, commonly referred to as a “hockey stick”.

Then, before incubation, newly shaken samples of coded (so that the microbiologist is blinded) toothpaste of various compositions with respect to both PVP-I and NaF is diluted 3-fold in sterile artificial saliva as commonly occurs in the course of human use of toothpaste during 1 min of brushing, and after brief vortex mixing, 10 μL used to wet blank sterile 10 mm diameter paper sensitivity-testing discs. These now-wetted discs are blotted briefly on sterile filter paper and carefully placed, wetted side-down, equidistantly onto the seeded surfaces of the agar plates, which are, in turn, now placed into the 37° C. incubator with a 5% CO₂/air atmosphere, agar side down. The disc-applied plates are inverted so as to obviate possible running of the agents or inocula across the humid agar surface and thereby “smearing” the surface. After 24 h incubation, the diameters of zones of no growth, measured by mm ruler or low-power microscope with ocular lens micrometer scale, are recorded on a spreadsheet. After all data are collected, they are decoded as to proportions of contents in the samples for later plotting on an isobologram.

Any bacteria strain from the streptococcus genus can be used. Preferably, we will use −80 C frozen strains of mutans streptococci, including S. mutans and S. sobrinus, the two prevalent mutans streptococcal human colonizers that are available. Strains that can be preferably used are any one of NCTC10449, ATCC25175 and ATCC27352. Other strains that can be used are any one of 10449S, LT-11, Ng8, and 6715-13WT. These other strains are described in Tanzer J M, Freedman M L, Fitzgerald R J, Larson R H. “Diminished virulence of glucan synthesis-defective mutants of Streptococcus mutans” Infect Immun. 1974 July; 10(1): 197-203. PMID: 4842127; Tanzer J M, Thompson A, Wen Z T, Burne R A. “Streptococcus mutans: Fructose transport, xylitol resistance, and virulence” J Dent Res 2006; 85:369-373. PMID: 16567561 PMCID:PMC2254530; Tanzer J M, Thompson A, Lang C, Cooper B, Hareng L, Gamer A, Reindl A, Pompejus M. “Caries inhibition by and safety of Lactobacillus paracasei DSMZ16671” J Dent Res 2010; 89:921-916. PMID:20519491; and Tanzer J M, Thompson A, Sharma K, Vickerman M M, Haase E M, Scannapieco F A. “Streptococcus mutans outcompetes S. gordonii in vivo” J Dent Res 2012; 91:513-519 (includes online Supplement). PMID:22431892.

### Statistical Analyses:

The synergistic effect can be determined from the plot found in FIG. 1.

If PVP-I and NaF manifest their growth-inhibiting effects independently (i.e. additively or indifferently), the plotted zones of clearing determined experimentally and representing the varying proportions of PVP-I and NaF will generally lie along the theoretical “Line of Indifference” depicted on the graph in FIG. 1, and the slope of the experimentally determined line for the combined agents will not be statistically significantly different from that of the Line of Indifference, anchored by the maximal growth inhibition effect of 100 portions of PVP-I/0 NaF on the upper left and by the maximal growth inhibition effect of 0 PVP-I/100 portions of NaF on the lower right.

By contrast, if PVP-I and NaF act synergistically to inhibit the growth of S. mutans, i.e. if the zones of no growth are larger than expected than if the agents were not indifferent to each other in effect, they will fall below and to the left of the Line of Indifference, and a line connecting those proportions will not correlate with the Line of Indifference. It will fall within the “Zone of Synergy”, and will be statistically different from the Line of Indifference.

Finally, if the data from the zones of clearing fall to the right and above the Line of Indifference, PVP-I and NaF must be acting antagonistically, and the values falling within the “Zone of Antagonism” and a line connecting those points will be statistically different from the Line of Indifference.

The test can identify the PVP-I toothpaste formulation with the greatest antibacterial efficacy.

### Test of De-Mineralization Inhibition and Re-Mineralization:

To ensure this formulation presents fluoride bioavailability compared to reference standards, the in vitro pH cycling model can determine the demineralization prevention and re-mineralization enhancement abilities. As reference standards, the following can be used: a) the currently marketed 1.1% NaF toothpaste (Elevate FluoriMax 5000®); and b) a PVP-I and fluoride-free toothpaste formulation.

The Knoop surface microhardness technique can be used to evaluate changes in the mineral status of human enamel samples.

### Test Substrate:

The substrate is human permanent incisors and molars. The teeth are cleaned of debris, polished and 4×4 mm enamel specimens prepared. Baseline surface microhardness determinations are made using a microhardness tester (2100 HT; Wilson Instruments, Norwood, MA, USA) with a Knoop diamond under 50-g load for 10 sec. Five indentations will be made at the center of the enamel surface. Enamel blocks with baseline surface microhardness between 300 and 400 KHN will be selected for the study. Eighteen blocks, with all the surfaces protected with an acid-resistant nail varnish except for the polished enamel surface, are used to test surface microhardness in each condition, by comparing the inventive formulations with the reference standards.

### Procedure:

In separate experiments, the blocks are subjected to 5 days of pH cycling. In short, twice daily, the blocks are treated with their assigned blinded toothpaste for 1 min. For each experiment, a fresh slurry is prepared just prior to each treatment by mixing one part toothpaste with three parts of a remineralizing solution (1.5 mM CaCl₂, 0.9 mM KH₂PO₄, 150 mM KCl, 0.050 ppm F− as NaF in 20 mM cacodylic buffer, pH 7.4). Between toothpaste treatments, the blocks are kept individually in a demineralizing solution (2.0 mM CaCl₂, 2.0 mM KH₂PO₄, 0.030 ppm F⁻ as NaF, in 75 mM acetate buffer, pH 4.3) for 3 h (20 ml/block). The blocks are stored in the re-mineralizing solution at all other times (approx. 20 h each day). This cycle is repeated daily for 5 days and then the enamel blocks remain in the remineralizing solution for 2 days until the analyses.

### Microhardness Determination:

After pH-cycling, surface microhardness is determined as described above by placing a further five indentations in close proximity to the sound enamel indentations. Knoop hardness numbers are then calculated. Surface microhardness is expressed as the % mean change in values between baseline and post treatment compared to controls.

### Analysis:

Analysis of variance (ANOVA) and paired t-test are used to compare % mean surface microhardness change between the treatments.

While this disclosure has been described as having an exemplary design, the present disclosure may be further modified within the spirit and scope of this disclosure. This application is therefore intended to cover any variations, uses, or adaptations of the disclosure using its general principles. Further, this application is intended to cover such departures from the present disclosure as come within known or customary practice in the art to which this disclosure pertains.

Other embodiments of the present disclosure will be apparent to those skilled in the art from consideration of the specification and practice of the embodiments disclosed herein. As used throughout the specification and claims, “a” and/or “an” may refer to one or more than one. Unless otherwise indicated, all numbers expressing quantities of ingredients, properties such as molecular weight, percent, ratio, reaction conditions, and so forth used in the specification and claims are to be understood as being modified in all instances by the term “about,” whether or not the term “about” is present. Accordingly, unless indicated to the contrary, the numerical parameters set forth in the specification and claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements. It is intended that the specification and examples be considered as exemplary only, with a true scope and spirit of the disclosure being indicated by the following claims. The foregoing embodiments are susceptible to considerable variation in practice. Accordingly, the embodiments are not intended to be limited to the specific exemplifications set forth hereinabove. Rather, the foregoing embodiments are within the spirit and scope of the appended claims, including the equivalents thereof available as a matter of law.

The patentees do not intend to dedicate any disclosed embodiments to the public, and to the extent any disclosed modifications or alterations may not literally fall within the scope of the claims, they are considered to be part hereof under the doctrine of equivalents.

It is to be understood that each component, compound, substituent or parameter disclosed herein is to be interpreted as being disclosed for use alone or in combination with one or more of each and every other component, compound, substituent or parameter disclosed herein.

It is also to be understood that each amount/value or range of amounts/values for each component, compound, substituent or parameter disclosed herein is to be interpreted as also being disclosed in combination with each amount/value or range of amounts/values disclosed for any other component(s), compounds(s), substituent(s) or parameter(s) disclosed herein and that any combination of amounts/values or ranges of amounts/values for two or more component(s), compounds(s), substituent(s) or parameters disclosed herein are thus also disclosed in combination with each other for the purposes of this description.

It is further understood that each range disclosed herein is to be interpreted as a disclosure of each specific value within the disclosed range that has the same number of significant digits. Thus, a range of from 1-4 is to be interpreted as an express disclosure of the values 1, 2, 3 and 4.

It is further understood that each lower limit of each range disclosed herein is to be interpreted as disclosed in combination with each upper limit of each range and each specific value within each range disclosed herein for the same component, compounds, substituent or parameter. Thus, this disclosure to be interpreted as a disclosure of all ranges derived by combining each lower limit of each range with each upper limit of each range or with each specific value within each range, or by combining each upper limit of each range with each specific value within each range.

Furthermore, specific amounts/values of a component, compound, substituent or parameter disclosed in the description or an example is to be interpreted as a disclosure of either a lower or an upper limit of a range and thus can be combined with any other lower or upper limit of a range or specific amount/value for the same component, compound, substituent or parameter disclosed elsewhere in the application to form a range for that component, compound, substituent or parameter.

1. An oral dentifrice composition comprising polyvinyl pyrrolidone-iodine (PVP-I) and a fluoride component at a concentration to provide at least 4500 ppm fluoride to the oral dentifrice composition.

2. The oral dentifrice composition of claim 1, wherein the PVP-I is present in an amount of about 0.5 wt. % to about 15 wt. % based on a total amount of the oral dentifrice composition.

3. The oral dentifrice composition of claim 1, wherein PVP-I provides iodine in an amount of about 0.05 wt. % to about 1.5 wt. % based on a total amount of the oral dentifrice composition.

4. The oral dentifrice composition of claim 1, wherein the fluoride component is a fluoride salt.

5. The oral dentifrice composition of claim 1, wherein the fluoride component is sodium fluoride (NaF), BiF₃, SnF₂, ZnF₂, KF, CaF₂, ZrF₄, sodium mono-fluorophosphate (Na₂FPO₃), hexafluorosilicic acid (H₂SiF₆), sodium hexafluorosilicate (Na₂SiF₆), or combinations thereof.

6. The oral dentifrice composition of claim 1, wherein the fluoride component is included in the composition in an amount from about 0.05 wt. % to about 10 wt. % based on a total amount of the oral dentifrice composition.

7. The oral dentifrice composition of claim 1, wherein the fluoride component is included in the composition in an amount sufficient to provide at least 4600 ppm fluoride to the oral dentifrice composition.

8. The oral dentifrice composition of claim 1, wherein the fluoride component is sodium fluoride.

9. The oral dentifrice composition of claim 1, wherein the fluoride component is sodium fluoride and has an average particle size (diameter) of less than 50 micrometers (microns) as measured by sieve analysis.

10. The oral dentifrice composition of claim 1, wherein the fluoride component is sodium fluoride where greater than 90% of the fluoride particles are less than 20±2 microns in diameter.

11. The oral dentifrice composition of claim 1, which is a paste, gel, cream, or powder.

12. The oral dentifrice composition of claim 1, further comprising an abrasive agent.

13. The oral dentifrice composition of claim 1, further comprising an abrasive agent selected from hydrated silica; alumina (including calcined aluminum oxide); diatomaceous earth; pumice; calcium carbonate; cuttlebone; and insoluble phosphates.

14. The oral dentifrice composition of claim 1, further comprising a tooth whitening or tooth bleaching component selected from peroxides; metal chlorites; and persulfates.

15. The oral dentifrice composition of claim 1, wherein the tooth whitening or tooth bleaching component is included in an effective amount.

16. The oral dentifrice composition of claim 1, further comprising one or more of potassium phosphate, a pH adjusting agent, surfactant, flavoring agent or sweetener, additional active ingredients, antibacterial agents, enzymes, an orally acceptable carrier, colorant, anticalculus agent, foaming agent, solubilizing agent, cleansing agent, humectant, antisensitivity agents, abrasive agent, tooth whitening or tooth bleaching component, and a hydrating agent.

17. The oral composition of claim 1, comprising one or more of the following:
a) A solvent to dissolve ingredients;
b) A hydrating agent;
c) An abrasive agent;
d) A humectant;
e) A pH adjusting agent;
f) A solubilizing or cleansing agent;
g) A flavoring agent;
h) A thickener or stabilizer;
i) A phosphate;
j) A foaming agent or thickener; and
k) A colorant.

18. A method for the treatment or prophylaxis of dental caries comprising applying the oral dentifrice composition of claim 1 to teeth in a mammal.

19. The method for the treatment or prophylaxis of dental caries according to claim 18, wherein the mammal is a human.

20. The oral composition of claim 1, wherein the fluoride component is at a concentration to provide at least 4800 ppm fluoride to the oral dentifrice composition.
